(19) United States
(12) Patent Application Publication
(10) Pub. No.: US 2020/0206017 A1
(43) Pub. Date: Jul. 2, 2020

(54) INCONTINENCE PAD FOR MEN

(71) Applicants: Celso Leal de Macedo, Sao Paulo (BR); Maria Belen Pombo de Macedo, Sao Paulo (BR)

(72) Inventors: Celso Leal de Macedo, Sao Paulo (BR); Maria Belen Pombo de Macedo, Sao Paulo (BR)

(21) Appl. No.: 16/232,111

(22) Filed: Dec. 26, 2018

(51) Int. Cl. A61F 5/453; A61F 5/44; A61F 13/56

(52) U.S. Cl. CPC A61F 5/453; A61F 5/4401; A61F 2013/15146; A61F 2005/4402; A61F 13/5611

(57) ABSTRACT

An incontinence pad, disposable and for daily use, which overlies the male pubic area, having a central opening that forms longitudinal folds and a space to support and accommodate the penis, with padded sides. There is an upper protective layer, developed in polypropylene and similar materials, arranged in the anterior portion up to two thirds of the pad, allowing the scrotum to be enclosed separately, avoiding possible irritation and inflammation by avoiding direct contact with the urine. The model in question offers more comfort and convenience to the user, in addition to discretion, as its thickness prevents it from being visible through the clothing.

The present invention is an improved incontinence pad, disposable and for daily use, which overlies the male pubic area, having a central opening that forms longitudinal folds and a space to support and accommodate the penis, with padded sides. There is an upper protective layer, developed in polypropylene and similar materials, arranged in the anterior portion up to two thirds of the pad, allowing the scrotum to be enclosed separately, avoiding possible irritation and inflammation by avoiding direct contact with the urine. The model in question offers more comfort and convenience to the user, in addition to discretion, as its thickness prevents it from being visible through the clothing. This application relates to improvements developed in Brazilian patent number MU8103426-1, granted on Jun. 28, 2011.

### BACKGROUND OF THE INVENTION

The purpose of this invention is to allow the user to have his penis and scrotum accommodated and enclosed by the pad area, avoiding inconveniences and discomfort that may be caused by urine leaks and secretions, depending on penis size, user movements, direct contact with urine, or any type of exposure.

The pad is fixed by adhesive tapes attached to the back of the pad directly to the polyethylene film or sheets of other impermeable materials that line the absorbent pad on the outside and are protected by protective papers or tape that are removed for use and attachment to the underwear.

Disposable unisex diapers for incontinence are already well known by users. The diapers, used by men and women alike, are made of a strip of cotton sheet covered on one side with a fabric sheet and on the other with a polyethylene sheet, and are placed on the human body between the legs, fastened up to the hip by adhesive tapes that allow for size adjustments.

Some pad models for women and others for men are also used, but such models do not provide as much comfort owing to two main factors: enclosure and accommodation of the male genitals and lack of description as to its use, as it is visible through the clothing.

### DESCRIPTION OF THE PRIOR ART

In order to place a novel product on the market, a search for previous products was carried out and the following prior art was revealed:

U.S. Pat. No. 5,380,310 discloses a disposable sanitary article to be used by men with incontinence, comprising an absorbent pad having an impermeable sheet attached to an outer side of said pad and a liquid-permeable sheet attached to an inner side of said pad, as well as a substantially rectangular substance with each longitudinal edge of the article being folded back into the liquid-permeable sheet to form longitudinal folds, with each longitudinal edge being attached to a central portion of the article at each end of both longitudinal folds, so as to form a half front, such that a portion of said longitudinal fold, found between each end of each longitudinal folds, is movable from a position close to the central part to a separate position, forming a pouch, wherein the liquid-permeable sheet is present in said pouch, in such a way that said sheet contacts the wearer's skin, and wherein said sanitary article is of size D in order to enclose men's genitals, but does not extend around the waist to absorb the fluid discharged by the genitals, and is not sized to enable the article to collect fecal matter from the adult male wearer.

Brazilian patent PI9505226-7 discloses an invention of a sanitary pad for men, comprising a small piece, of anatomical model and made of absorbent and disposable material, with adhesive tape to be glued on the front inner part of the underwear, in order to prevent small secretions and penile discharge from staining the underwear.

Brazilian patent PI9505282-8 discloses a sanitary napkin for men, comprising a tube, of anatomical model and closed at the front, made of absorbent and disposable material, to be worn on the penis, in order to prevent small secretions and penile discharge from staining the underwear.

Brazilian patent PI0704842-4 discloses a sanitary pad for male incontinence comprised of a reservoir and its body, both in the lower part, connected to a cylindrical body having a self-adhesive flap in the middle, and at its upper end a crimped elastic to both fix and offer greater malleability to the penis of the man suffering from urinary incontinence. The present model also gives comfort and safety to men affected by this disorder, since, with the product, they will feel more virile in everyday situations where the use of disposable diapers would cause great discomfort.

Brazilian patent PI9508733-8 discloses a useful absorbent product as a male incontinence device. The product can be converted from a two-dimensional structure for storage to a three-dimensional structure for use. There are folds forming pleats located within the product to allow the inner part to expand from a pleated to an expanded structure, and the outer part of the product; and secured in order to prevent substantial expansion. The product can be handled from a substantially flat two-dimensional structure for storage to a three-dimensional cup-like structure for use. The manufacturing methods of such absorbent products are also described

Brazilian patent PI0520697-9 discloses a product for male incontinence”. The present invention relates to a male guard (10) for incontinence. The guard is divided by a transverse partition line (25) at the front part (18) with a front transverse edge (20) and a back region (22) with a rear transverse edge (24). The guard (10) tapers towards the back transverse edge (24). The surface (26) of the guard (10) turned to the underwear (10) comprises a first strip (28) and a second strip (30) of adhesive means. The position and dimensions of the first strip (28) and the second strip (30) of adhesive means provide improved fixation of the guard (10) to the wearer's underwear.

Brazilian patent PI9809108-5 under the title “Disposable absorbent device for male users and use thereof” discloses a disposable absorbent device for male users containing an outer layer and an absorbent wad, in which the outer layer part located next to the wearer's body has an opening, preferably of long and narrow shape, equipped with elastics to adjust and wrap the penis and/or penis and scrotum of the user. The devices of the invention are particularly suitable for absorbency of incontinent males, including bedridden patients, and may also adapt to the needs of infants.

Brazilian patent PI9803474-0 under the title “Urine pad for men” discloses a urine pad for men is disclosed, composed of a pouch of a laminated sheet comprising an inner sheet, an outer sheet, and an absorbent core between them, and a pair of elastic elements which are provided along the opening. By holding and pushing both ends of the elastic elements with the fingers, they are deformed by buckling to open the aperture. When a penis is inserted into the opening, and the fingers are then released, the penis is properly pressed through the restoring elastic forces of the elastic elements in order to prevent the penis from slipping off easily. Since the compressive force resulting from the buckling deformation of the elastic elements is very small, a great deal of pressure is placed on the penis. In addition, the urine-receiving cushion can be placed with just one hand.

According to the searched prior art, the mentioned cases do not have any similarity nor the advantages of the object of the present invention, for which reason it is considered that there are no technical or legal impediments to obtaining the claimed patent.

In most of the cases, disclosed in the prior art, the male genitals remain upright, next to the accumulated discharged matter and urine.

In this described invention the male genitals are placed downward, without any contact with the discharge, or with other parts of the body and urine.

### Main Problems of the Prior Art

In most of the cases, disclosed in the prior art, the male genitals remain upright, next to the accumulated discharged matter and urine. Several issues are not properly addressed and remain without solution such as:
Discomfort;
Penis strangulation;
Inadequate penis accommodation;
Direct contact with urine;
Rashes and allergies;
Injuries;
Size standardization;
Contact with groin, causing irritation.

### Major Advantages of the Invention

Therefore, the invention presented discloses an improvement in the model of incontinence pad for men, with the purpose of offering more comfort and safety to the user, as well as to prevent leaks and embarrassments, comprising several benefits, namely:
Shaped with cutout spaces, pleats and longitudinal folds that form a penile enclosure;
Penis and scrotum are well accommodated and enclosed by the absorbent area;
Prevention of leaks;
Comfort and safety to the user;
Avoiding direct contact with urine;
Preventing rashes, inflammation and irritations;
Allowing free movement of the user, avoiding discomfort;
Easily attachable to underwear and to be worn;
Easy to dispose of after use;
Can be manufactured in different sizes;
Ease of production

### DRAWINGS

The objects and advantages of the present invention will become apparent from the description taken in conjunction with the accompanying drawings, wherein:

FIG. 1—Shows a rear view of the object in question;

FIG. 2—Shows a lateral view of the object in question;

FIG. 3—Shows a frontal view of the object in question;

FIG. 4—Shows an upper view of the object in question;

FIG. 5—Shows a cross-section view of the object in question;

FIG. 6—Shows an illustrative view of the object in question, illustrating the adhesive tape being withdrawn for adhesion and attachment of the pad to the underwear;

FIG. 7—Shows an illustrative view of the object in question, illustrating the pad being attached to the underwear;

FIG. 8—Shows an illustrative view of the object in question, illustrating the pad attached to the underwear;

FIG. 9—Shows an illustrative view of the object in question, illustrating the accommodation near the penile region;

FIG. 10—Shows an illustrative view of the object in question being used.

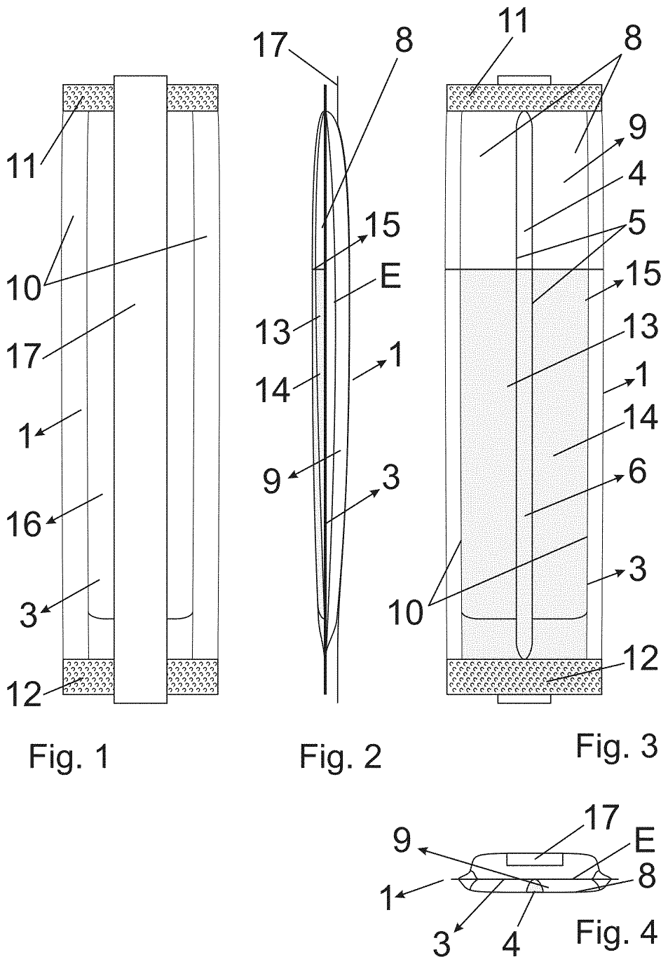

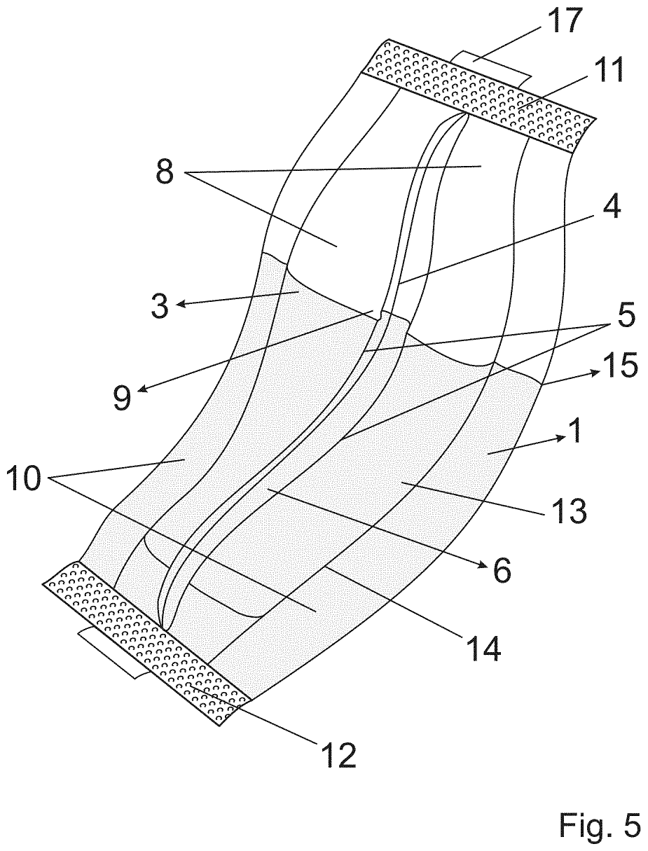

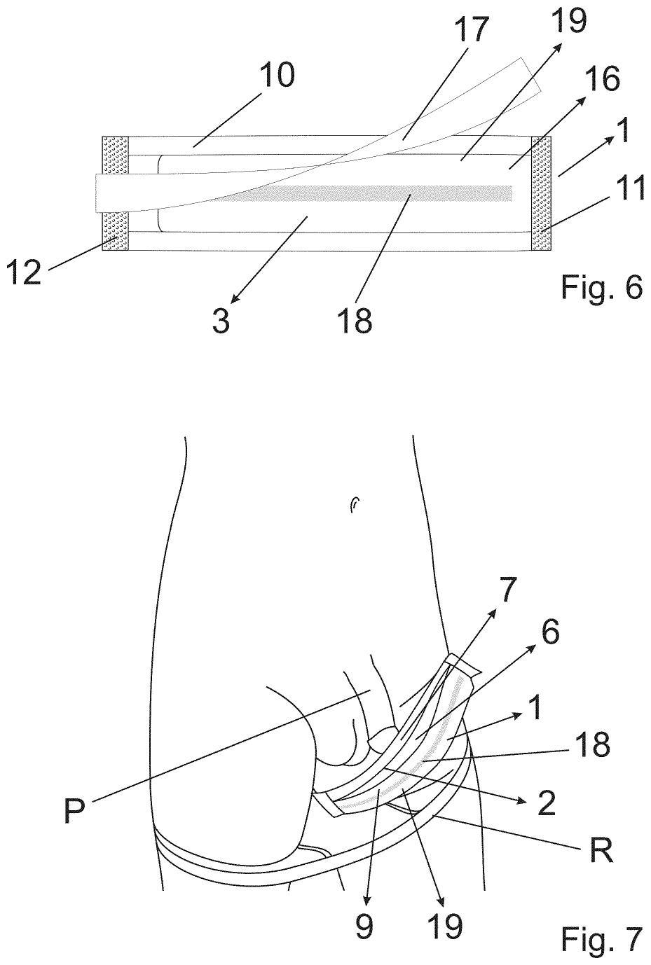

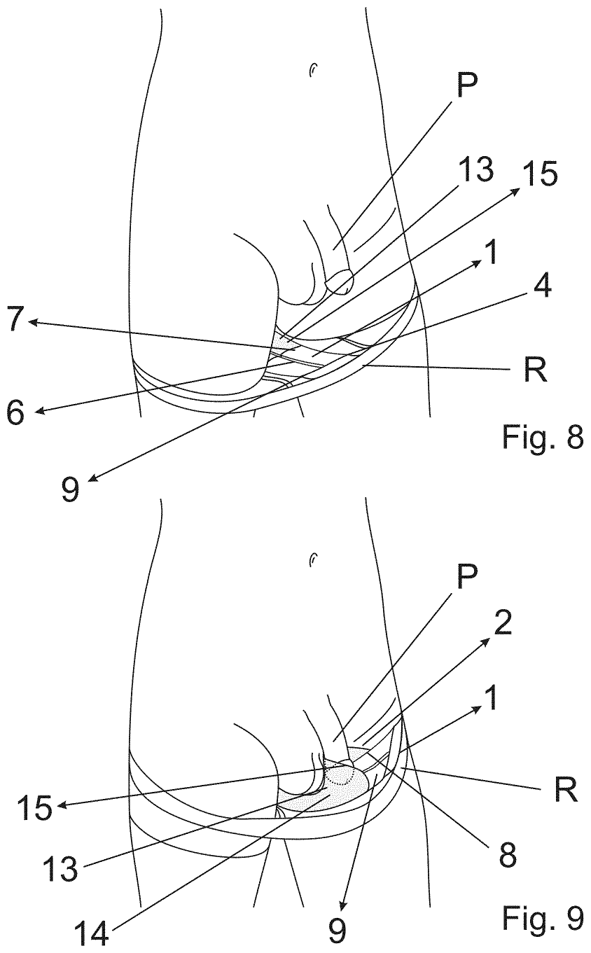

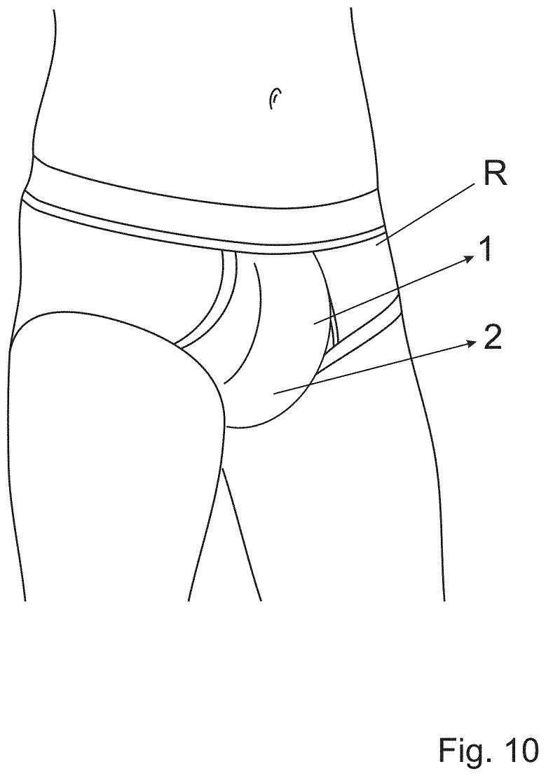

### DETAILED DESCRIPTION OF THE INVENTION

According to the illustration and its details, the invention is formed by a disposable pad for men (1), of thin thickness (E), placed on the man's genitals (2), characterized by a rectangular shape and constitution (3), a central open space (4), forming longitudinal pleats (5) and a central compartment (6) for penile support and enclosure (7), with padded sides (8); having also absorbent layers (9), which are joined and sealed with glue on the sides (10) and upper (11) and lower sealing (12), and a trimmed protective layer (13), developed in polypropylene and similar materials (14) arranged up to up to two thirds of the pad (15).

The trimmed protective layer (13) prevents direct contact of the penis (P) and urine with other regions, preventing rashes and irritations.

The pad (1) also has in its back portion (14) an adhesive tape (15) with glue (16) that, when removed (17), allows it to be fixed to the clothing (R).

The main body of this pad has two longitudinal folds sealed at the ends, forming a central open space and increase the internal area of the pad. This central open space forms an internal space in the middle of the pad to accommodate of the testicular pouch of the user, allowing for its perfect accommodation.

In the main body of the pad, occupying only ¾ of the pad area, a layer of green SMS Hydrophobic Polypropylene SW is applied, an insulating material, which creates an impermeable compartment in the pad and in which the penis should be inserted downwards, in order to limit its movement and determine the urinary loss area. The main function of this compartment is to protect the rest of the man's body, such as groin, scrotum and genital folds, from humidity, thus preventing the formation of rashes caused by urine.

The male sanitary pad is composed of absorbent materials, an acrylic polymer cellulose sheet (GEL), which absorbs more than 400 ml of urine per pad. This layer is covered on the upper side by a polypropylene layer, intended to protect the absorbent material, and on the lower side by a layer of impermeable polypropylene film, which prevents liquid leakage. A layer of thermoplastic adhesive is applied onto the film protected by a silicone paper tape which, when removed, allows the application of the product on the underwear. All materials are non-toxic.

In describing the invention, reference has been made to preferred embodiments and illustrative advantages of the invention. Those skilled in the art, however, and familiar with the instant disclosure of the invention, may recognize additions, deletions, modifications, substitutions, and other changes which will fall within the purview of the subject invention.

1. Incontinence pad for men comprising a disposable pad for men (1), of thin thickness (E), placed on the man's genitals (2), characterized by a rectangular shape and constitution (3), a central open space (4), forming longitudinal pleats (5) and a central compartment (6) for penile support and enclosure (7), with padded sides (8); having also absorbent layers (9), which are joined and sealed with glue on the sides (10) and upper (11) and lower sealing (12), and a trimmed protective layer (13), developed in polypropylene and similar materials (14) arranged up to up to two thirds of the pad (15).

2. Incontinence pad for men, according to claim 1, characterized by a trimmed protective layer (13), prevents direct contact of the penis (P) and urine with other regions, preventing rashes and irritations.

3. Incontinence pad for men, according to claim 1 characterized by the pad (1) also having in its back portion (14) an adhesive tape (15) with glue (16) that, when removed (17), allows it to be fixed to the clothing (R).
